(19) United States
(12) Patent Application Publication
(10) Pub. No.: US 2026/0334897 A1
(43) Pub. Date: Oct. 29, 2026

(54) RADIOGRAPHY SYSTEM AND IMAGING UNIT

(71) Applicant: FUJIFILM Corporation, Tokyo (JP)

(72) Inventors: Hisatsugu HORIUCHI, Kanagawa (JP); Masayoshi MATSUURA, Kanagawa (JP); Kyohei MORI, Kanagawa (JP); Naoyuki NISHINO, Kanagawa (JP); Takeyasu KOBAYASHI, Kanagawa (JP)

(73) Assignee: FUJIFILM Corporation, Tokyo (JP)

(21) Appl. No.: 19/227,184

(22) Filed: Jun. 3, 2025

(30) Foreign Application Priority Data
Jun. 6, 2024 (JP) 2024-092509

(51) Int. Cl. A61B 6/00; A61B 6/04; A61B 6/40; A61B 6/58

(52) U.S. Cl. CPC A61B 6/4405; A61B 6/0407; A61B 6/40; A61B 6/582

(57) ABSTRACT

A radiography system includes an imaging unit used for radiography and including a traveling mechanism that travels on a floor, and a radiation source or a radiation detector, and a subject support apparatus that supports a subject of radiography and that includes an engaging portion which attachably and detachably engages with the imaging unit.

### CROSS-REFERENCE TO RELATED APPLICATIONS

The present application claims priority under 35 U.S.C. § 119 to Japanese Patent Application No. 2024-092509 filed on Jun. 6, 2024. The above application is hereby expressly incorporated by reference, in its entirety, into the present application.

### BACKGROUND

### 1. Technical Field

The present disclosure relates to a radiography system and an imaging unit.

### 2. Description of the Related Art

JP2021-176505A discloses a mobile medical apparatus system comprising a mobile X-ray apparatus in which an X-ray source is mounted on a traveling mechanism such as an electric cart, and a charging station for charging a battery of the mobile X-ray apparatus. The battery of the mobile X-ray apparatus can be charged by docking the mobile X-ray apparatus at the charging station.

### SUMMARY

An imaging unit such as the mobile X-ray apparatus according to JP2021-176505A includes a traveling mechanism and thus, is less stable than a fixed imaging unit. Thus, it is desired to store the imaging unit in a stable state while the imaging unit is not used for imaging.

However, since there are various apparatuses such as a decubitus imaging table and an upright imaging table in an imaging room in which the imaging unit is installed, it is often difficult to secure a dedicated storage location for storing the imaging unit in a stable state.

The disclosed technology provides a radiography system and an imaging unit for enabling storage of an imaging unit including a traveling mechanism in a stable state without providing a dedicated storage location.

According to an aspect of the disclosed technology, there is provided a radiography system comprising an imaging unit used for radiography and including a traveling mechanism that travels on a floor, and a radiation source or a radiation detector, and a subject support apparatus that supports a subject of radiography and that includes an engaging portion which attachably and detachably engages with the imaging unit.

In the aspect, two imaging units including a radiation source unit including the radiation source and a panel unit including the radiation detector may be provided as the imaging unit.

In the aspect, the engaging portion may be disposed at a position higher than a centroid of the imaging unit in a vertical direction.

In the aspect, the subject support apparatus may be at least one of an imaging table or a holding apparatus capable of being held by the subject in performing the radiography.

In the aspect, the subject support apparatus may be fixed in a room in which the imaging unit is installed.

In the aspect, a connector capable of electrically connecting to the imaging unit in a state where the engaging portion and the imaging unit engage with each other may be provided.

In the aspect, the connector may be integrated with the engaging portion.

In the aspect, the connector may function as a charging connector of a battery mounted on the imaging unit.

In the aspect, the traveling mechanism may be an electric traveling mechanism that electrically travels on a floor.

In the aspect, the electric traveling mechanism may have an automatic traveling function of automatically moving to a designated position.

In the aspect, the electric traveling mechanism may move to a connection position for connection to the connector in a case where a condition set in advance is satisfied.

In the aspect, the electric traveling mechanism may execute calibration related to a traveling control after moving to a reference position set in advance.

In the aspect, the connector may be movable.

In the aspect, the subject support apparatus may have a function of notifying the imaging unit of a current position of the connector.

In the aspect, the connector may be connectable at a plurality of positions within a certain range.

In the aspect, the connector may be connected to a wiring line projecting from under a floor.

In the aspect, in connecting to the connector in a bottom portion of the traveling mechanism, the connector may be disposed at a position higher than a floor surface.

In the aspect, in a case where the subject support apparatus includes a movable portion that includes a position obstructing connection to the imaging unit in a movable range, the subject support apparatus may include a collision prevention mechanism that prevents a collision between the movable portion and the imaging unit.

According to another aspect of the disclosed technology, there is provided an imaging unit comprising a traveling mechanism that travels on a floor, and a radiation source or a radiation detector used for radiography, in which the imaging unit attachably and detachably engages with an engaging portion included in a subject support apparatus that supports a subject of radiography.

According to the disclosed technology, an imaging unit including a traveling mechanism can be stored in a stable state without providing a dedicated storage location.

### BRIEF DESCRIPTION OF THE DRAWINGS

FIG. 1 is a diagram illustrating an example of a schematic configuration of a radiography system.

FIG. 2 is a diagram illustrating an example of a connection state between an imaging unit and a decubitus imaging table.

FIG. 3 is a diagram illustrating an example of a connection state between the imaging unit and a holding apparatus.

FIGS. 4A and 4B are diagrams illustrating an example of a connection state between the decubitus imaging table and the imaging unit in a case where an engaging portion moves.

FIG. 5 is a diagram illustrating an example of a movable connector.

FIG. 6 is a diagram illustrating an example of a line-shaped connector.

FIG. 7 is a block diagram illustrating a summary of a functional configuration of the imaging unit.

FIG. 8 is a diagram illustrating an example of a controller of an electric traveling mechanism.

FIG. 9 is a diagram illustrating an example of automatic traveling.

FIG. 10 is a flowchart illustrating an example of a control of the imaging unit.

FIG. 11 is a flowchart illustrating another example of the control of the imaging unit.

FIG. 12 is a diagram illustrating an example of a control of the decubitus imaging table.

FIG. 13 is a diagram illustrating a modification example of the connector.

FIG. 14 is a diagram illustrating an example of collision prevention processing of the holding apparatus.

FIG. 15 is a diagram illustrating a variation on the collision prevention processing.

FIG. 16 is a diagram illustrating an example of collision prevention processing of the decubitus imaging table.

### DETAILED DESCRIPTION

### First Embodiment

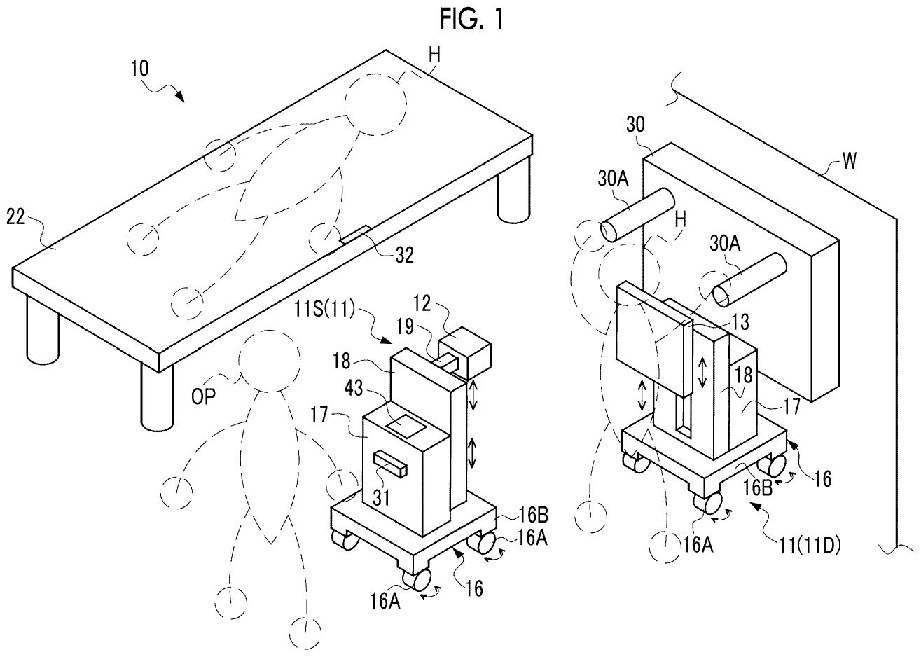

FIG. 1 is a schematic diagram illustrating an example of a configuration of a radiography system 10 that performs radiography of a subject H. The radiography system 10 comprises a radiation source unit 11S and a panel unit 11D. For example, these units are provided in a radiography room and are operated by an operator OP such as a medical radiologist. The radiation source unit 11S includes an emission portion 12. The emission portion 12 is an example of a “radiation source” according to the disclosed technology. The emission portion 12 includes an X-ray tube that generates X-rays which are an example of radiation, an emission field limiter that limits an emission range of the X-rays, and the like. The radiation source unit 11S is also provided with a high-voltage generator that generates a high voltage to be supplied to the X-ray tube, and the like.

The panel unit 11D includes a detection panel 13 that is an example of a radiation detection panel. For example, the detection panel 13 is a flat panel detector having a detection surface on which pixels that detect X-rays and output electric signals corresponding to an incidence amount of the X-rays are two-dimensionally arranged. The detection panel 13 is an example of a “radiation detector” according to the disclosed technology.

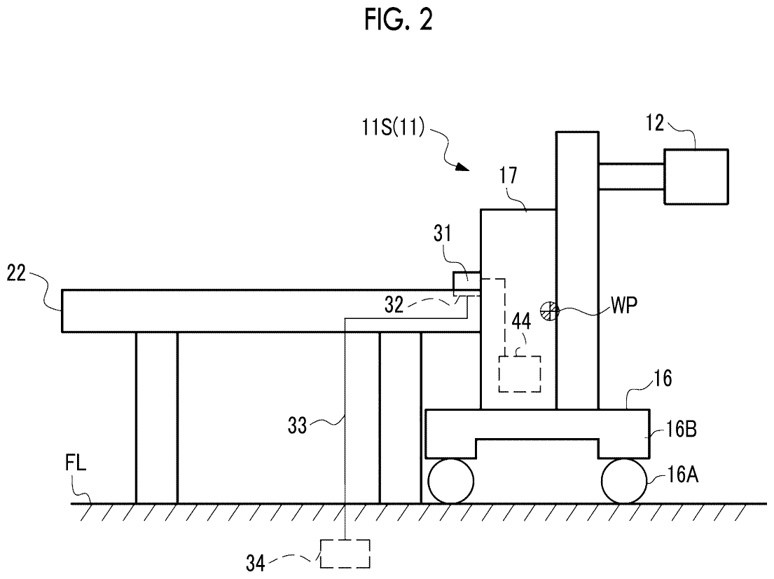

The radiography system 10 captures an X-ray image of the subject H by emitting X-rays from the emission portion 12 and detecting the X-rays transmitted through the subject H via the detection panel 13. Each of the radiation source unit 11S and the panel unit 11D includes a carriage portion 16 including wheels 16A. For example, the carriage portion 16 includes a base portion 16B that is a body part having a rectangular planar shape. The wheels 16A are provided at four corners of the base portion 16B, and the carriage portion 16 is of a four-wheel type. For example, each wheel 16A is a revolving wheel that revolves about a revolution axis extending in a height direction (referred to as a vertical direction) orthogonal to a rotation axis in rotating to travel. The carriage portion 16 can be manually caused to travel on a floor FL (refer to FIG. 2 and the like). Thus, the operator OP can move installation locations of the radiation source unit 11S and the panel unit 11D by manually causing the carriage portion 16 to travel. The carriage portion 16 is an example of a “traveling mechanism” that travels on the floor FL.

The radiation source unit 11S includes a body part 17 and a movable mechanism for changing a height of the emission portion 12. The movable mechanism is composed of a movable portion 18 of which a height changes with respect to the body part 17, and an arm 19 of which a height changes with respect to the movable portion 18. The emission portion 12 is provided at a tip end of the arm 19. The emission portion 12 is provided at a free end of the arm 19. While illustration is not provided, the arm 19 is provided with a swing mechanism that changes an emission direction by tilting the emission portion 12. The body part 17 incorporates various electric components, and an operation panel 43 is provided on its upper surface.

Like the radiation source unit 11S, the panel unit 11D also includes a body part 17 and a movable mechanism that changes a height of the detection panel 13. The movable mechanism of the panel unit 11D is also composed of a movable portion 18 of which a height changes with respect to the body part 17, and an arm (not illustrated) of which a height with respect to the movable portion 18 changes, and is the same as the movable mechanism of the radiation source unit 11S. The detection panel 13 is provided at a free end of the arm. Like the body part 17 of the radiation source unit 11S, the body part 17 also incorporates various electric components, and the operation panel 43 is provided on its upper surface.

While the body parts 17, the movable portions 18, and the arms 19 in each of the radiation source unit 11S and the panel unit 11D are parts having different incorporated components, shapes, and the like, differences between both are not important in the disclosed technology. Thus, both of them will be designated by the same reference numerals. Both of the radiation source unit 11S and the panel unit 11D are examples of an “imaging unit” according to the disclosed technology. Hereinafter, both of the radiation source unit 11S and the panel unit 11D will be referred to as imaging units 11 unless distinction therebetween is required.

As illustrated in FIG. 1, for example, in imaging the subject H in a standing posture, the radiation source unit 11S and the panel unit 11D are disposed to face each other with the subject H interposed therebetween. In performing imaging using a decubitus imaging table 22 on which the subject H can be placed in a decubitus posture, for example, a portable detection panel (not illustrated) called an electronic cassette and the radiation source unit 11S are used in combination. The decubitus imaging table 22 may incorporate a detection panel. In this case, the incorporated detection panel may be used.

As illustrated in FIG. 2, in the radiography system 10, the decubitus imaging table 22 includes an engaging portion 32 that attachably and detachably engages with the radiation source unit 11S. An engaging portion 31 that engages with the engaging portion 32 is provided in the body part 17 of the radiation source unit 11S. For example, the engaging portion 32 and the engaging portion 31 are fitting portions such as a protruding portion and a recessed portion, and both can be fitted with each other to be connected. For example, in storing the radiation source unit 11S when the radiation source unit 11S is not in use, the engaging portion 32 and the engaging portion 31 are engaged with each other. Accordingly, the radiation source unit 11S is supported by the decubitus imaging table 22. The decubitus imaging table 22 is an apparatus that supports the subject and thus, has a robust configuration and is fixed to the floor FL of an imaging room using a bolt or the like so as not to be inadvertently moved. The decubitus imaging table 22 is an example of a “subject support apparatus” that supports the subject H according to the disclosed technology.

In the radiation source unit 11S, the engaging portion 31 is disposed at a position higher than a centroid WP of the radiation source unit 11S in the vertical direction. The engaging portion 32 of the decubitus imaging table 22 is also provided at a position corresponding to the engaging portion 31.

The engaging portion 31 and the engaging portion 32 function as connectors that electrically connect the radiation source unit 11S and the decubitus imaging table 22 to each other, in addition to a support function of mechanically supporting the radiation source unit 11S. That is, each of the engaging portion 31 and the engaging portion 32 is integrated with an electrical connector. Specifically, the engaging portion 31 and the engaging portion 32 include conductive connection terminals in addition to the mechanical fitting portions. For example, as illustrated in FIG. 2, the engaging portion 31 of the radiation source unit 11S is connected to a battery 44 mounted on the radiation source unit 11S and functions as a charging connector. The engaging portion 32 of the decubitus imaging table 22 is connected to a power outlet 34 for power supply provided in the imaging room through a cable 33. By engaging the engaging portion 31 and the engaging portion 32 with each other, the battery 44 can be charged from the power outlet 34 through the engaging portion 32 and the engaging portion 31.

For example, the power outlet 34 is disposed under the floor FL so as not to cause an obstruction and can be accessed by removing a part of the floor FL. The cable 33 projects from under the floor FL and is pulled out to an upper surface of the floor FL through an outlet provided in a part of the floor FL.

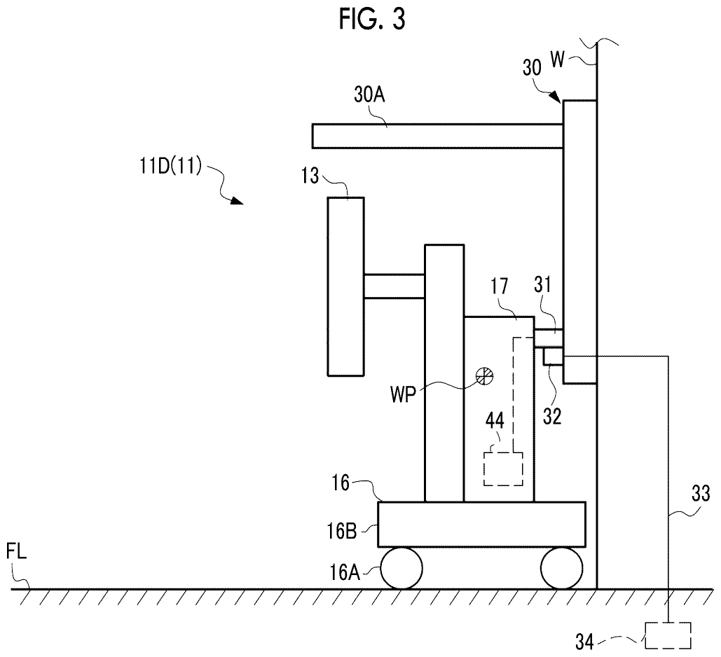

As illustrated in FIG. 3, the engaging portion 31 and the engaging portion 32 are also provided on the panel unit 11D side. As illustrated in FIGS. 1 and 3, a holding apparatus 30 is provided on a wall W of the imaging room. The holding apparatus 30 is a device for causing the subject H subjected to imaging in an upright posture to hold the holding apparatus 30 to maintain the posture of the subject H. The holding apparatus 30 includes two holding rods 30A that can be held by the subject H with hands during imaging. The holding apparatus 30 is fixed to the wall W using a bolt or the like. The body part 17 of the panel unit 11D is also provided with the same engaging portion 31 as the radiation source unit 11S. The holding apparatus 30 is also provided with the same engaging portion 32 as the engaging portion 32 of the decubitus imaging table 22. The holding apparatus 30 is also an apparatus that supports the subject H like the decubitus imaging table 22, and thus, has a robust configuration and is fixed to the wall W of the imaging room so as not to be inadvertently moved. The holding apparatus 30 is an example of the “subject support apparatus” that supports the subject H according to the disclosed technology.

The engaging portion 31 of the panel unit 11D and the engaging portion 32 of the holding apparatus 30, like those in the radiation source unit 11S illustrated in FIG. 2, function as connectors that electrically connect the panel unit 11D and the holding apparatus 30 to each other, in addition to a support function of mechanically supporting the panel unit 11D. The point of functioning as a charging connector is also the same as that in the radiation source unit 11S.

As described above, the radiography system 10 according to the disclosed technology is used for radiography and comprises the imaging unit 11 that includes the traveling mechanism (for example, the carriage portion 16) which travels on the floor FL, and the radiation source (for example, the emission portion 12) or the radiation detector (for example, the detection panel 13), and the subject support apparatus (for example, the decubitus imaging table 22 and the holding apparatus 30) that supports the subject of radiography and includes the engaging portion 32 which attachably and detachably engages with the imaging unit 11.

According to the above, the imaging unit 11 including the traveling mechanism as illustrated by the carriage portion 16 can be stored in a stable state without providing a dedicated storage location. That is, the imaging unit 11 including the traveling mechanism such as the carriage portion 16 may be inadvertently moved or may be unstable even in a standalone state, compared to a fixed imaging apparatus fixed to a predetermined location in the imaging room. While it is preferable to secure a dedicated storage location to store the imaging unit 11 in a stable state, it may be difficult to secure such a space. Meanwhile, the subject support apparatus that supports the subject, such as the decubitus imaging table 22 and the holding apparatus 30, is installed in the imaging room. The subject support apparatus has a function of supporting the subject H and thus, is highly stable, as described above. In the radiography system 10 according to the disclosed technology, the subject support apparatus is provided with the engaging portion 32 that engages with the imaging unit 11. Since the imaging unit 11 can be stored in a state of being engaged with the engaging portion 32, the imaging unit 11 can be stored in a stable state without providing a dedicated storage location.

In the above embodiment, the imaging unit 11 constituting the radiography system 10 includes two imaging units 11 including the radiation source unit 11S and the panel unit 11D. That is, the engaging portion 31 is provided in both of the imaging units 11, and the radiography system 10 comprises the decubitus imaging table 22 and the holding apparatus 30, each of which includes the engaging portion 32 that engages with the engaging portion 31. Thus, both of the two imaging units 11 can be stored in a stable state. The engaging portion 31 may be provided in only one of the two imaging units 11.

In the above embodiment, the engaging portion 32 that engages with the engaging portion 31 of the imaging unit 11 is disposed at a position higher than the centroid WP of the imaging unit 11 in the vertical direction. Thus, since the imaging unit 11 is supported at a position higher than the centroid WP, the imaging unit 11 can be in a more stable state than that supported at a position lower than the centroid WP.

In the above embodiment, the subject support apparatus is at least one of an imaging table (for example, the decubitus imaging table 22) or the holding apparatus 30 that can be held by the subject during radiography. Apparatuses such as the decubitus imaging table 22 and the holding apparatus 30 are often installed in the imaging room. Thus, by using these apparatuses, a new subject support apparatus may not have to be added. The imaging table includes not only the decubitus imaging table 22 but also an upright imaging table, and the engaging portion 32 may be provided in the upright imaging table. The subject support apparatus may be a handrail or the like provided on the wall W of the imaging room.

In the above embodiment, the subject support apparatus such as the decubitus imaging table 22 and the holding apparatus 30 is fixed to a room such as the imaging room in which the imaging unit 11 is installed. Thus, very high stability is achieved, and the imaging unit 11 can be stored in a more stable state than the imaging unit 11 that is not fixed.

In the above embodiment, a connector that can be electrically connected to the imaging unit 11 in a state where the engaging portion 32 and the imaging unit 11 engage with each other is provided. Thus, charging or the like can be performed using the connector. In the above embodiment, the engaging portion 32 and the connector are integrated with each other, and the engaging portion 32 functions as the connector. Thus, a configuration can be simplified compared to that in a case where the engaging portion 32 and the connector are separated from each other. The engaging portion 32 and the connector may be separated from each other.

In a case where the subject support apparatus such as the holding apparatus 30 and the decubitus imaging table 22 includes the movable mechanism, power supply is required for the subject support apparatus itself. Thus, the power outlet 34 and power supply and other wiring lines or the like are provided near the subject support apparatus. Alternatively, as illustrated in FIGS. 2 and 3, in a case where the power outlet 34 and the wiring lines are provided in a space under the floor, an outlet for the wiring lines projecting from under the floor is provided near the subject support apparatus. Thus, in a case where the subject support apparatus is a location at which the engaging portion 32 is provided, existing wiring lines provided in advance for the subject support apparatus can be used as wiring lines of the electrical connector. In providing the electrical connector in this manner, the existing wiring lines can be effectively used by providing the engaging portion 32 in the subject support apparatus. This achieves an advantage of not requiring installation of a new wiring line.

In the above embodiment, the engaging portion 32 as the connector functions as the charging connector of the battery 44 mounted on the imaging unit 11. Thus, the imaging unit 11 can be charged while being stored.

In the above embodiment, the engaging portion 32 as the connector is connected to the cable 33 that is an example of the wiring lines projecting from under the floor FL. Thus, since the wiring lines are hidden under the floor, the number of wiring lines routed around the subject support apparatus engaged with the imaging unit 11 are reduced. Accordingly, the wiring lines are unlikely to obstruct traveling of the imaging unit 11.

### Modification Example of Connector

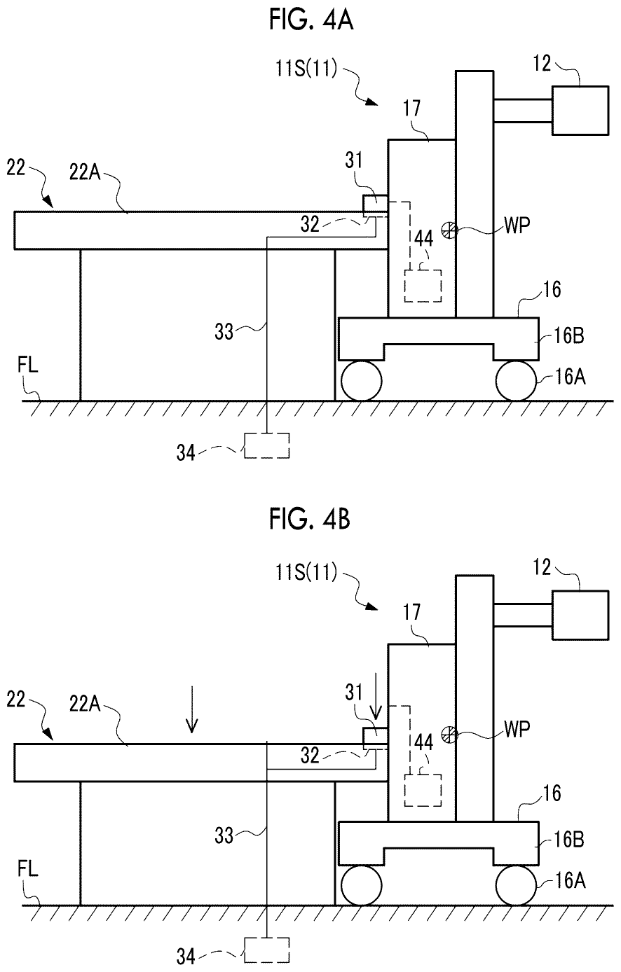

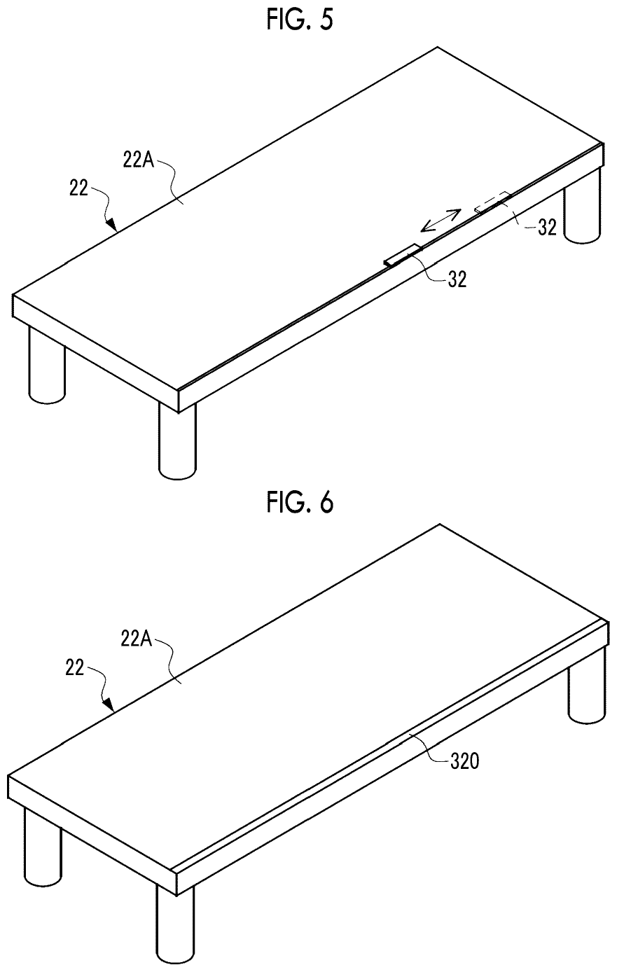

As illustrated in FIGS. 4A and 4B, a top plate 22A of the decubitus imaging table 22 may be raised or lowered between the position illustrated in FIG. 4A and the position illustrated in FIG. 4B. In a case where the engaging portion 32 is provided on the top plate 22A, the engaging portion 32 functioning as the connector is also raised or lowered in accordance with raising and lowering of the top plate 22A. As illustrated in FIG. 5, in the decubitus imaging table 22, the engaging portion 32 functioning as the connector may move in a horizontal direction on the top plate 22A. In a case where the connector is movable, a connection position with respect to the imaging unit 11 can be changed. In this case, the engaging portion 31 of the imaging unit 11 is also movable, and a position of the engaging portion 31 is adjusted in accordance with movement of the engaging portion 32.

As illustrated in FIG. 6, an engaging portion 320 as the connector can be connected at a plurality of positions within a certain range. In the engaging portion 320 illustrated in FIG. 6, for example, a metal piece as an electrical terminal extends in a line shape, and the engaging portion 320 can be connected to the engaging portion 31 at any position within the range. According to this, since the connection position of the imaging unit 11 can be changed, high usability is achieved.

### Second Embodiment

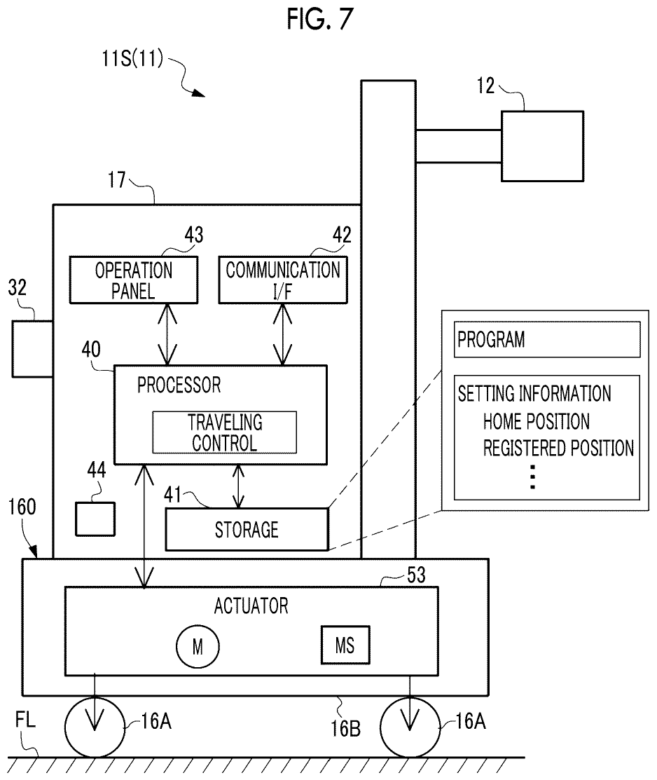

While the above embodiment illustrates the carriage portion 16 as an example of a manual traveling mechanism, the carriage portion 16 may be an electric traveling mechanism that electrically travels on the floor FL, like a carriage portion 160 of a second embodiment illustrated in FIG. 7. A carriage portion 160 is an example of an “electric traveling mechanism” according to the disclosed technology. The imaging unit 11 illustrated in FIG. 7 is the radiation source unit 11S, and an example of providing the electric traveling mechanism in the radiation source unit 11S will be described.

The radiation source unit 11S comprises, in addition to the battery 44, a processor 40, a storage 41, a communication interface (I/F) 42, and an operation panel 43. For example, the processor 40 is composed of a central processing unit (CPU) and a memory such as a random access memory (RAM) and functions as various controllers by executing a program loaded into the memory. The processor 40 not only controls the whole radiation source unit 11S in an integrated manner but also executes an emission control of the X-rays of the emission portion 12 and a traveling control of the carriage portion 160.

The storage 41 is a data storage that is composed of a hard disk drive, a solid state drive, a non-volatile memory, and the like and stores not only the program but also data of various types of setting information.

The communication I/F 42 is, for example, a wireless communication portion and executes wireless communication with another imaging unit 11 such as the panel unit 11D.

The operation panel 43 is an operator for inputting an operation instruction and is composed of, for example, a touch panel display.

The carriage portion 160 incorporates an electric circuit for electric traveling and electric components such as an actuator 53. For example, the carriage portion 160 is of a four-wheel type including four wheels 16A, and each wheel 16A is a revolving wheel. These points indicate that the carriage portion 160 is the same as the carriage portion 16. For example, all of the wheels 16A are drive wheels that are electrically rotated. Two of the four wheels 16A may be steering wheels, and the remaining two may be drive wheels.

The actuator 53 includes a motor M that drives the wheels 16A. The motor M includes a motor for causing the wheels 16A to rotate to travel and a motor for causing the wheels 16A to revolve. A measurement sensor MS measures rotation amounts, rotation directions, revolution directions, revolution angles, and the like of the wheels 16A and a moving direction, a moving amount, and the like of the carriage portion 160. Measurement values measured by the measurement sensor MS are input into the processor 40. For example, the measurement sensor MS is composed of a rotary encoder and an acceleration sensor. The processor 40 executes a traveling control such as self position estimation of the carriage portion 160 based on the received measurement values.

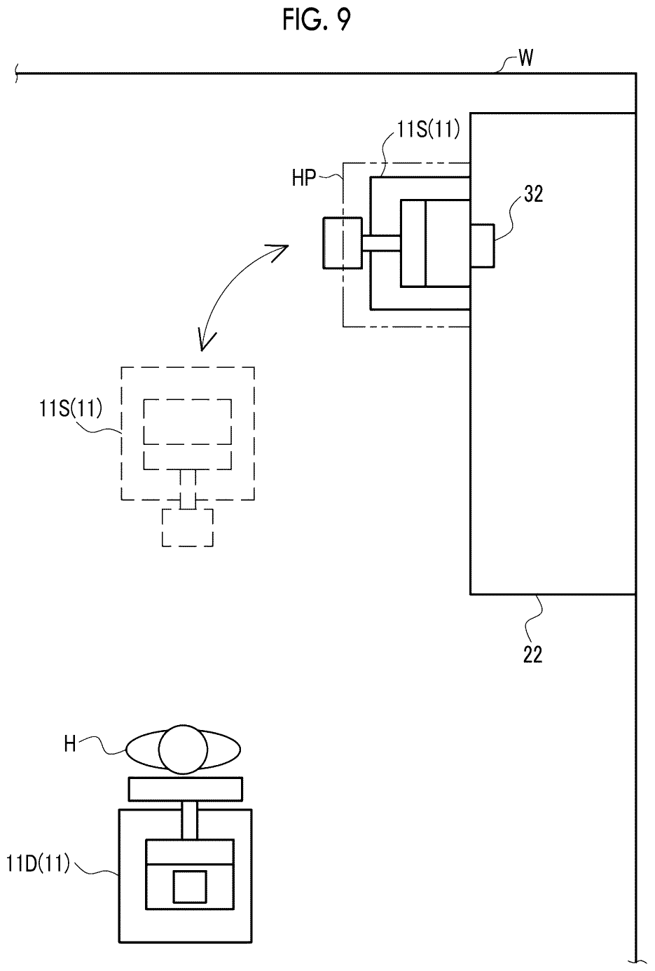

In the self position estimation, the processor 40 acquires the measurement values from the measurement sensor MS and, for example, obtains the moving amount of the carriage portion 160 from a home position HP (refer to FIG. 9 and the like). For example, the home position HP is a position registered as a reference position of the radiation source unit 11S in the imaging room. As described above, the measurement sensor MS measures the measurement values such as the rotation amounts, the rotation directions, the revolution amounts, and the revolution angles of the wheels 16A. The processor 40 executes the self position estimation of estimating a relative self position of the carriage portion 160 with respect to the home position HP by accumulating the measured values. Such self position estimation is self position estimation based on a so-called odometry technique. By performing such self position estimation, the carriage portion 160 can be automatically returned to the home position HP.

A traveling control of automatically moving the carriage portion 160 to not only the home position HP but also a registered position set in advance can also be performed. For example, usage such as registering an imaging position having a high frequency of use in advance and automatically moving the imaging unit to the imaging position is considered.

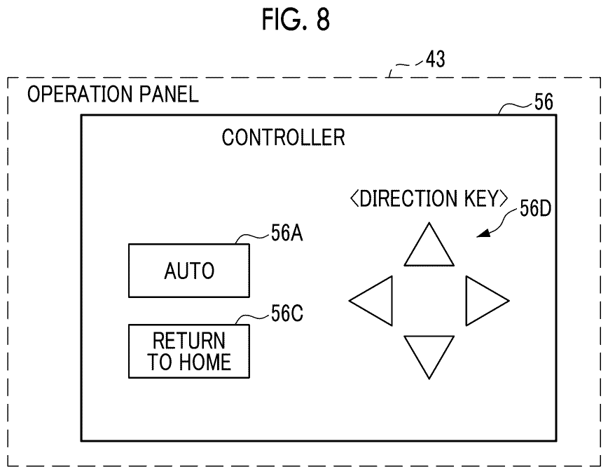

FIG. 8 is an example of a screen, displayed on the operation panel 43, of a controller 56 for operating the carriage portion 160. For example, an operation instruction including a moving instruction for the carriage portion 160 is input through the controller 56.

The controller 56 is provided with an auto button 56A, a return button 56C, and a direction key 56D. The auto button 56A is an operation button for inputting a moving instruction for automatically moving to a registered position registered in advance into the processor 40. The return button 56C is an operation button for inputting a return instruction for returning the carriage portion 160 to the home position HP set in advance as an initial position of the carriage portion 160. The direction key 56D is an operation button for inputting a moving instruction for moving the carriage portion 160 in any direction of four directions.

The auto button 56A and the return button 56C are operation buttons for causing the carriage portion 160 to autonomously execute a traveling control set in advance. Meanwhile, the direction key 56D is an operation button for executing a simple traveling control of providing an instruction for only a direction and moving by an operation amount in the direction provided by the instruction. The direction key 56D is of a so-called dead man's type that continues outputting an operation signal during an operation and stops outputting the operation signal in a case where the operation stops. This is safer than a case where the operation signal is output even in a state where a hand is released from the direction key 56D.

The traveling control of the carriage portion 160 executed by the processor 40 will be described with reference to FIG. 9. For example, as illustrated in FIG. 9, it is assumed that the initial position of the radiation source unit 11S is the home position HP, and the radiation source unit 11S is at the home position HP. For example, the home position HP is a connection position at which the radiation source unit 11S is connected to the decubitus imaging table 22 through the engaging portion 32. In this state, for example, the operator OP operates the direction key 56D to move the radiation source unit 11S to a position facing the panel unit 11D by causing the carriage portion 160 to travel. In moving from the home position HP, the processor 40 measures the moving amount with reference to the home position HP through the measurement sensor MS.

In a case where imaging is finished and the operator OP operates the return button 56C, the processor 40 controls the carriage portion 160 to automatically travel to the home position HP. Accordingly, the radiation source unit 11S returns to the home position HP. A traveling control of automatically moving the carriage portion 160 to not only the home position HP but also a registered position set in advance can also be performed by operating the auto button 56A.

While the second embodiment describes the radiation source unit 11S as an example of the imaging unit 11, the panel unit 11D may also be provided with the electric traveling mechanism by providing the carriage portion 160 in the same manner.

In the second embodiment, for example, the traveling mechanism is the electric traveling mechanism as illustrated by the carriage portion 160. Since electric traveling is enabled, movement of the imaging unit 11 is facilitated compared to that in manual traveling.

In the second embodiment, for example, the electric traveling mechanism illustrated by the carriage portion 160 has an automatic traveling function of automatically moving to a designated position such as the home position HP. According to this, since the imaging unit 11 can be simply caused to automatically travel to the designated position, high usability is achieved.

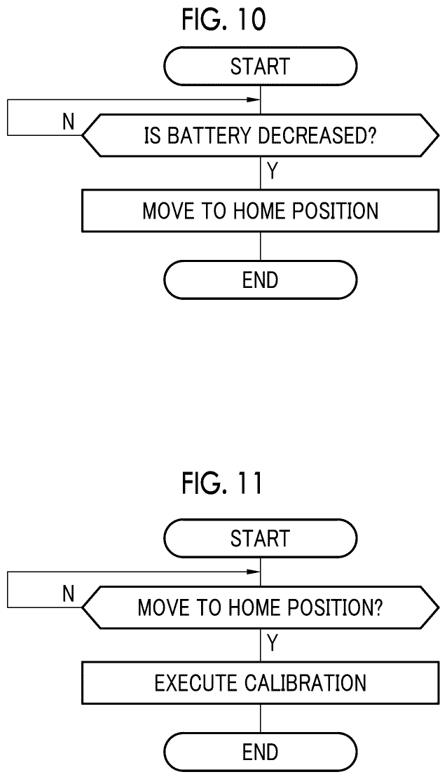

The processor 40 may perform the controls illustrated in FIGS. 10 and 11 using the carriage portion 160, in addition to the traveling control. FIG. 10 illustrates a control of moving the carriage portion 160 to the connection position for connecting to the engaging portion 32 as the connector in a case where a condition set in advance is satisfied. For example, the condition set in advance is a condition that a remaining capacity of the battery 44 of the radiation source unit 11S is less than a capacity set in advance. In this case, the carriage portion 160 moves to the home position HP, which is the connection position with respect to the engaging portion 32, under control of the processor 40. The radiation source unit 11S is charged by electrically connecting the radiation source unit 11S to the engaging portion 32 as the charging connector. Such a control can prevent the remaining capacity of the battery 44 from being used up at a position other than a chargeable position. The condition set in advance may be a condition other than a decrease in the remaining capacity of the battery 44 and may be, for example, a time point set in advance other than an operation time, such as night-time. In this case, the carriage portion 160 moves to the home position HP under control of the processor 40 in a case where the set time point is reached.

FIG. 11 illustrates an example of executing calibration related to the traveling control of the self position estimation after moving to the home position HP. As described above, in the self position estimation of the carriage portion 160, the processor 40 executes the self position estimation based on the cumulative moving amount based on the home position HP as the reference position. In the self position estimation, accuracy is decreased in a case where measurement error of the measurement sensor MS is accumulated. Thus, it is preferable to execute calibration related to the traveling control, such as updating the reference position in the measurement sensor MS. Therefore, after moving to the home position HP which is the reference position set in advance, the carriage portion 160 executes calibration related to the traveling control under control of the processor 40 by, for example, resetting the measurement values of the measurement sensor MS. Accordingly, performing a control of temporarily canceling the measurement error at the reference position can improve accuracy of the self position estimation.

### Other Modification Examples

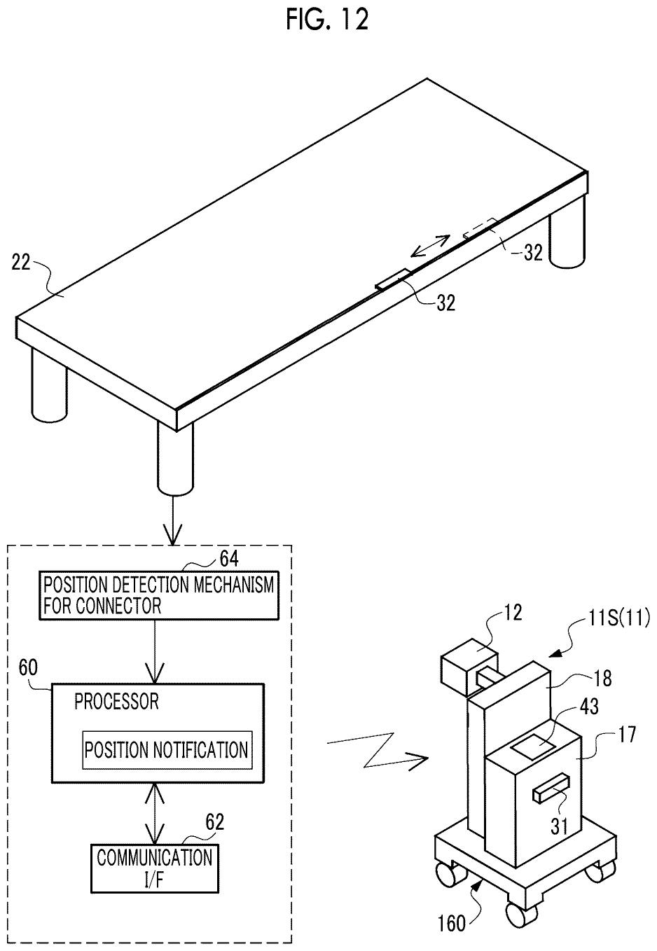

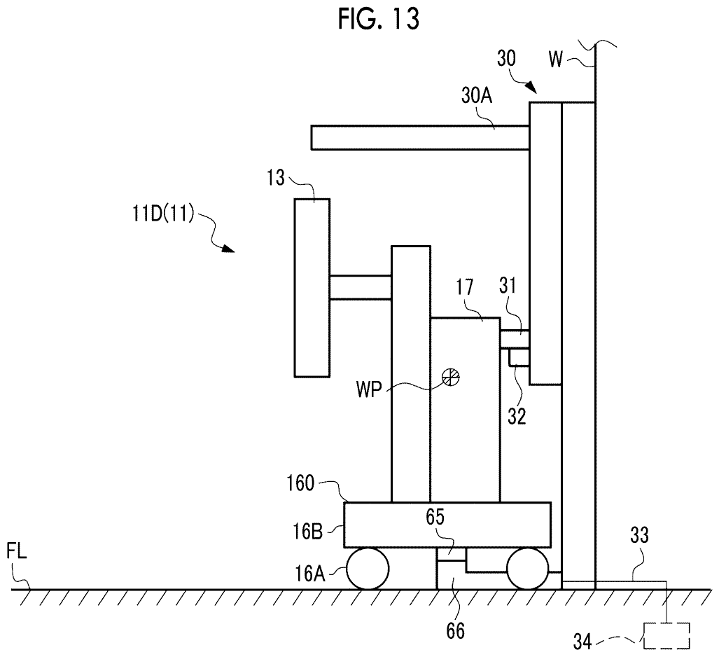

Various modifications other than the above can be made to the radiography system 10. FIG. 5 illustrates an example in which the engaging portion 32 as the connector is movable. In this case, as illustrated in FIG. 12, it is preferable that the subject support apparatus has a function of notifying the imaging unit 11 of the current position of the engaging portion 32. For example, as illustrated in FIG. 12, the decubitus imaging table 22 is provided with a processor 60, a communication I/F 62, and a position detection mechanism 64. The processor 60 is composed of a CPU, a memory, and the like, like the processor 40, and the communication I/F 62 is a wireless communication portion like the communication I/F 42. The position detection mechanism 64 detects the current position of the engaging portion 32 as the connector. The position detection mechanism 64 is composed of a potentiometer that outputs a signal corresponding to the position of the engaging portion 32, an encoder that optically detects the position of the engaging portion 32 using a photo sensor, and the like.

The processor 60 notifies the imaging unit 11 illustrated by, for example, the radiation source unit 11S of the current position of the engaging portion 32. The imaging unit 11 receives the current position of the engaging portion 32, obtains the connection position with respect to the engaging portion 32, and corrects a position to which the imaging unit 11 moves. Accordingly, even in automatically traveling to the designated position via the electric traveling mechanism such as the carriage portion 160, a moving destination can be appropriately corrected in accordance with the current position of the movable engaging portion 32.

As illustrated in FIG. 13, various modifications can also be made to a form of a connector 66 for electrical connection. As illustrated in FIG. 13, in connecting the imaging unit 11 to the connector 66 in a bottom portion of the carriage portion 160 which is the traveling mechanism, it is preferable to dispose the connector 66 at a position higher than a floor surface. In the example illustrated in FIG. 13, the holding apparatus 30 as the subject support apparatus is provided with the connector 66 for electrical connection separately from the engaging portion 32 that supports the imaging unit 11. The connector 66 is provided on the floor FL. Meanwhile, the imaging unit 11 also includes a connector 65 for electrical connection separately from the engaging portion 31, and the connector 65 is provided in a bottom portion of the carriage portion 160.

The carriage portion 160 is provided with a clearance between the bottom portion and the floor surface so as to travel on a slightly uneven floor surface. Thus, in a case where the connector 65 is provided in the bottom portion of the carriage portion 160, it is preferable to dispose the connector 66 on the holding apparatus 30 side at a position higher than the floor surface as illustrated by, for example, an aspect of protruding from the floor surface in FIG. 13.

The connector 65 and the connector 66 may be connected in a contact manner or a contactless manner using an electric field. Even in a case where a contactless manner is used, such a configuration is effective because a distance between the connector 65 and the connector 66 is important in terms of power supply efficiency and the like.

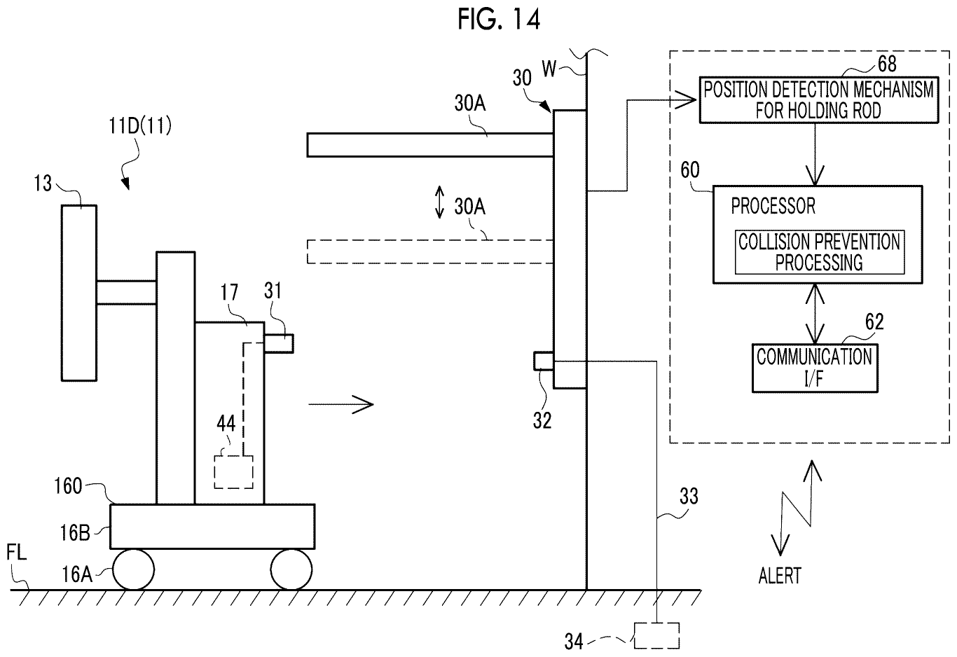

As illustrated in FIG. 14, in a case where the holding apparatus 30 as the subject support apparatus includes a movable portion (for example, the holding rod 30A) including a position obstructing connection to the imaging unit 11 in a movable range, the holding apparatus 30 includes a collision prevention mechanism that prevents collision between the movable portion and the imaging unit 11.

For example, the movable range of the holding rod 30A is between a position indicated by a solid line and a position indicated by a dotted line. In a case where the imaging unit 11 and the holding apparatus 30 are connected to each other through engaging between the engaging portion 31 and the engaging portion 32, the position indicated by the dotted line obstructs connection to the imaging unit 11. In this state, trying to connect the imaging unit 11 causes collision between the holding rod 30A, which is the movable portion, and the imaging unit 11. Therefore, the holding apparatus 30 includes the processor 60, and the processor 60 functions as the collision prevention mechanism by executing collision prevention processing. A position detection mechanism 68 for the holding rod 30A outputs the current position of the holding rod 30A to the processor 60. The position detection mechanism 68 is the same as the position detection mechanism 64 illustrated in FIG. 12 and is composed of a potentiometer or the like that outputs a signal corresponding to the position of the holding rod 30A.

For example, in a case where the processor 60 receives notification indicating connection from the imaging unit 11 through the communication I/F 62, the processor 60 issues an alert to the imaging unit 11 through the communication I/F 62 in a case where the holding rod 30A and the imaging unit 11 may collide with each other. In a case where the imaging unit 11 receives such an alert, the imaging unit 11 stops moving to the connection position with respect to the holding apparatus 30. Accordingly, a collision between the imaging unit 11 and the holding rod 30A is prevented.

In the processor 60 of the holding apparatus 30, approach of the imaging unit 11 may be detected using an approach detection mechanism such as a photo sensor and a camera, instead of detecting approach of the imaging unit 11 based on notification from the imaging unit 11.

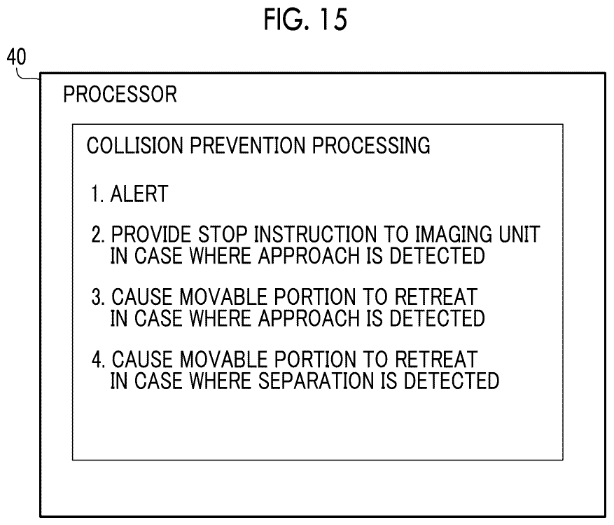

As illustrated in FIG. 15, various types of processing other than the alert are also considered as the collision prevention processing. For example, the processor 60 detects the imaging unit 11 approaching the holding apparatus 30 and, in a case where this may result in a collision, outputs a stop instruction to the imaging unit 11 in addition to or instead of the alert. By providing the stop instruction to the imaging unit 11 via the processor 60 of the holding apparatus 30, an effect of collision prevention is improved compared to that based on the alert.

In a case where the processor 60 detects approach of the imaging unit 11, the processor 60, as the collision prevention processing, may cause the holding rod 30A to retreat to a retreat position indicated by a solid line in a case where the holding rod 30A is at a position indicated by a dotted line in FIG. 14.

In a case where the processor 60 detects the imaging unit 11 separating from the holding apparatus 30, the processor 60, as the collision prevention processing, may unconditionally cause the holding rod 30A to retreat to the retreat position indicated by the solid line in FIG. 14. Accordingly, in a case where the imaging unit 11 is connected, a state where the holding rod 30A is always at the retreat position can be secured.

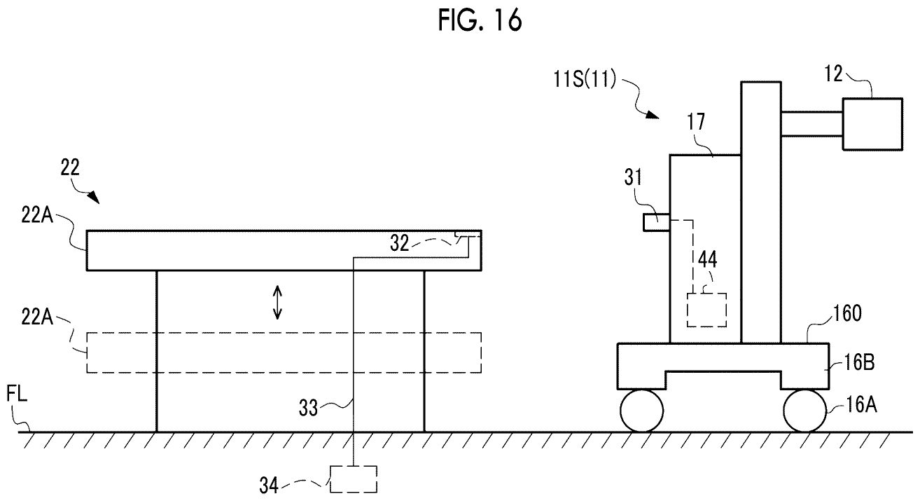

While the example illustrated in FIG. 14 illustrates the holding apparatus 30 as an example, the collision prevention processing may also be required in the decubitus imaging table 22 illustrated in FIG. 16. For example, in a case where the top plate 22A of the decubitus imaging table 22 is raised or lowered, the top plate 22A may collide with the imaging unit 11 depending on a position of the top plate 22A. Even in this case, it is preferable that the processor 60 of the decubitus imaging table 22 executes the collision prevention processing illustrated in FIGS. 14 and 15.

While the above embodiments describe the engaging portion 32 and the engaging portion 31 as being mechanically connected to each other, the engaging portion 32 and the engaging portion 31 may be magnetically connected to each other using a magnet or the like. The engaging portion 32 and the engaging portion 31 can be provided with various aspects such as an aspect of being connected to each other by a hook, regardless of the aspect of being fitted with each other to be connected.

For the autonomous traveling control of the electric traveling mechanism illustrated by the carriage portion 160 as an example, a simultaneous localization and mapping (SLAM) technique for autonomously traveling while performing the self position estimation and map creation at the same time may be used.

In the traveling mechanism illustrated in the above embodiments, the form of the wheels is for illustrative purposes, and various modifications can be made. For example, the number of front wheels and/or the number of rear wheels may be changed independently of each other. For example, a combination of one front wheel and two rear wheels or a combination of two front wheels and three rear wheels may be adopted. For example, sizes and shapes may be changed between the front wheels and the rear wheels.

In the above embodiments, various types of hardware described below can be used as hardware of the processor 40, the processor 60, and the like. The various types of hardware include, in addition to a CPU that is a general-purpose processor functioning as various processing portions by executing software (a program), a programmable logic device (PLD) such as a field-programmable gate array (FPGA) that has a circuit configuration changeable after manufacture, a dedicated electric circuit such as an application specific integrated circuit (ASIC) that has a circuit configuration dedicatedly designed to execute specific processing, and the like.

The above various types of processing may be executed by one of the various types of hardware or may be executed by a combination of two or more pieces of hardware of the same type or different types (for example, a plurality of FPGAs and a combination of a CPU and an FPGA). A plurality of processing portions may be composed of one piece of hardware. Examples of the plurality of processing portions composed of one piece of hardware include a form of using hardware that implements functions of the whole system including the plurality of processing portions in one integrated circuit (IC) chip, like a system on chip (SOC).

The various processing portions are composed of one or more of the various types of hardware.

More specifically, electric circuits (circuitry) obtained by combining circuit elements such as semiconductor elements can be used as various hardware structures.

The disclosed technology also includes, in addition to a program for operating the imaging unit, a computer-readable storage medium (a universal serial bus (USB) memory, a digital versatile disc (DVD)-read only memory (ROM), or the like) that stores the program in a non-transitory manner. The disclosed technology can also be applied to a program and a program product.

Content of description and content of illustration shown above are detailed description for parts according to the disclosed technology and are merely an example of the disclosed technology. For example, description related to the above configurations, functions, actions, and effects is description related to examples of configurations, functions, actions, and effects of the parts according to the disclosed technology. Thus, unnecessary parts may be removed, new elements may be added, or the parts may be replaced with each other in the content of description and the content of illustration shown above without departing from the gist of the disclosed technology. Particularly, description related to common technical knowledge or the like not required to be described for embodying the disclosed technology is omitted in the content of description and the content of illustration shown above in order to avoid complication and facilitate understanding of the parts according to the disclosed technology.

The technology according to the following appendices can be perceived from the above description.

### Appendix 1

A radiography system comprising:
an imaging unit including a traveling mechanism that travels on a floor, and a radiation source or a radiation detector used for radiography; and
a subject support apparatus that supports a subject of radiography and that includes an engaging portion which engages with the imaging unit.

### Appendix 2

The radiography system according to Appendix 1,
in which two imaging units including a radiation source unit including the radiation source and a panel unit including the radiation detector are provided as the imaging unit.

### Appendix 3

The radiography system according to Appendix 1 or 2,
in which the engaging portion is disposed at a position higher than a centroid of the imaging unit in a vertical direction.

### Appendix 4

The radiography system according to any one of Appendices 1 to 3,
in which the subject support apparatus is at least one of an imaging table or a holding apparatus capable of being held by the subject in performing the radiography.

### Appendix 5

The radiography system according to any one of Appendices 1 to 4,
in which the subject support apparatus is fixed in a room in which the imaging unit is installed.

### Appendix 6

The radiography system according to any one of Appendices 1 to 5,
in which a connector capable of electrically connecting to the imaging unit in a state where the engaging portion and the imaging unit engage with each other is provided.

### Appendix 7

The radiography system according to Appendix 6,
in which the connector is integrated with the engaging portion.

### Appendix 8

The radiography system according to Appendix 6 or 7,
in which the connector functions as a charging connector of a battery mounted on the imaging unit.

### Appendix 9

The radiography system according to any one of Appendices 1 to 8,
in which the traveling mechanism is an electric traveling mechanism that electrically travels on a floor.

### Appendix 10

The radiography system according to Appendix 9,
in which the electric traveling mechanism has an automatic traveling function of automatically moving to a designated position.

### Appendix 11

The radiography system according to Appendix 10,
in which the electric traveling mechanism moves to a connection position for connection to the connector in a case where a condition set in advance is satisfied.

### Appendix 12

The radiography system according to Appendix 10 or 11,
in which the electric traveling mechanism executes calibration related to a traveling control after moving to a reference position set in advance.

### Appendix 13

The radiography system according to any one of Appendices 6 to 12,
in which the connector is movable.

### Appendix 14

The radiography system according to Appendix 13,
in which the subject support apparatus has a function of notifying the imaging unit of
a current position of the connector.

### Appendix 15

The radiography system according to any one of Appendices 6 to 14,
in which the connector is connectable at a plurality of positions within a certain range.

### Appendix 16

The radiography system according to any one of Appendices 6 to 15,
in which the connector is connected to a wiring line projecting from under a floor.

### Appendix 17

The radiography system according to any one of Appendices 6 to 16,
in which, in connecting to the connector in a bottom portion of the traveling mechanism,
the connector is disposed at a position higher than a floor surface.

### Appendix 18

The radiography system according to any one of Appendices 1 to 17,
in which, in a case where the subject support apparatus includes a movable portion that includes a position obstructing connection to the imaging unit in a movable range,
the subject support apparatus includes a collision prevention mechanism that prevents a collision between the movable portion and the imaging unit.

### Appendix 19

An imaging unit comprising:
a traveling mechanism that travels on a floor; and
a radiation source or a radiation detector used for radiography,
in which the imaging unit attachably and detachably engages with an engaging portion included in a subject support apparatus that supports a subject of radiography.

In the present specification, “A and/or B” is synonymous with “at least one of A or B”. That is, “A and/or B” may mean only A, only B, or a combination of A and B. In the present specification, the same approach as “A and/or B” also applies to an expression of three or more matters connected with “and/or”.

All documents, patent applications, and technical standards described in the present specification are incorporated in the present specification by reference to the same extent as those in a case where individual documents, patent applications, and technical standards are specifically and individually indicated to be incorporated by reference.

What is claimed is:

1. A radiography system comprising:
an imaging unit used for radiography and including a traveling mechanism that travels on a floor, and a radiation source or a radiation detector; and
a subject support apparatus that supports a subject of radiography and that includes an engaging portion which attachably and detachably engages with the imaging unit.

2. The radiography system according to claim 1,
wherein two imaging units including a radiation source unit including the radiation source and a panel unit including the radiation detector are provided as the imaging unit.

3. The radiography system according to claim 1,
wherein the engaging portion is disposed at a position higher than a centroid of the imaging unit in a vertical direction.

4. The radiography system according to claim 1,
wherein the subject support apparatus is at least one of an imaging table or a holding apparatus capable of being held by the subject in performing the radiography.

5. The radiography system according to claim 1,
wherein the subject support apparatus is fixed in a room in which the imaging unit is installed.

6. The radiography system according to claim 1,
wherein a connector capable of electrically connecting to the imaging unit in a state where the engaging portion and the imaging unit engage with each other is provided.

7. The radiography system according to claim 6,
wherein the connector is integrated with the engaging portion.

8. The radiography system according to claim 6,
wherein the connector functions as a charging connector of a battery mounted on the imaging unit.

9. The radiography system according to claim 8,
wherein the traveling mechanism is an electric traveling mechanism that electrically travels on a floor.

10. The radiography system according to claim 9,
wherein the electric traveling mechanism has an automatic traveling function of automatically moving to a designated position.

11. The radiography system according to claim 10,
wherein the electric traveling mechanism moves to a connection position for connection to the connector in a case where a condition set in advance is satisfied.

12. The radiography system according to claim 10,
wherein the electric traveling mechanism executes calibration related to a traveling control after moving to a reference position set in advance.

13. The radiography system according to claim 6,
wherein the connector is movable.

14. The radiography system according to claim 13,
wherein the subject support apparatus has a function of notifying the imaging unit of a current position of the connector.

15. The radiography system according to claim 6,
wherein the connector is connectable at a plurality of positions within a certain range.

16. The radiography system according to claim 6,
wherein the connector is connected to a wiring line projecting from under a floor.

17. The radiography system according to claim 6,
wherein, in connecting to the connector in a bottom portion of the traveling mechanism,
the connector is disposed at a position higher than a floor surface.

18. The radiography system according to claim 1,
wherein, in a case where the subject support apparatus includes a movable portion that includes a position obstructing connection to the imaging unit in a movable range,
the subject support apparatus includes a collision prevention mechanism that prevents a collision between the movable portion and the imaging unit.

19. An imaging unit comprising:
a traveling mechanism that travels on a floor; and
a radiation source or a radiation detector used for radiography,
wherein the imaging unit attachably and detachably engages with an engaging portion included in a subject support apparatus that supports a subject of radiography.
